(19) United States
(12) Patent Application Publication
(10) Pub. No.: US 2025/0302523 A1
(43) Pub. Date: Oct. 2, 2025

(54) MULTI-FUNCTIONAL SURGICAL CAUTERY DEVICE, SYSTEM AND METHOD OF USE

(71) Applicant: Z Surgical LLC, Sherman Oaks, CA (US)

(72) Inventor: Gabriel Zada, Encino, CA (US)

(21) Appl. No.: 18/863,177

(22) PCT Filed: May 3, 2023
(86) PCT No.: PCT/US2023/020866

§ 371 Date: Nov. 5, 2024

Related U.S. Application Data
(60) Provisional application No. 63/364,255, filed on May 5, 2022.
(60) Provisional application No. 63/497,069, filed on Apr. 19, 2023.

(51) Int. Cl. A61B 18/12; A61B 18/00; A61B 34/20; A61B 90/00; A61B 90/30

(52) U.S. Cl. CPC A61B 18/1206; A61B 34/20; A61B 90/30; A61B 2018/00178; A61B 2018/00595; A61B 2018/00642; A61B 2018/00678; A61B 2018/0072; A61B 2018/00767; A61B 2018/00875; A61B 2034/2048; A61B 2090/061; A61B 2090/064; A61B 2090/309; A61B 2090/3941

(57) ABSTRACT

A surgical cautery device, system, and method of use may apply bipolar and/or sesquipolar electrocautery to target tissue via instruments with other primary surgical functions. The surgical cautery device and system may include one or more sensors configured to detect when a surgical cautery device is in use. The surgical cautery device and system may also include one or more sensors configured to detect a location of the surgical cautery device or instrument. The surgical cautery device and system may also include a processor and software capable of varying current based on characteristics of the system. The device and system may also include adaptors capable of allowing multiple surgical instruments to be plugged into or wirelessly connected to the system.

### PRIORITY CLAIM AND INCORPORATION BY REFERENCE

The present application is a National Phase Application of PCT/US2023/020866 filed May 3, 2023, titled MULTI-FUNCTIONAL SURGICAL CAUTERY DEVICE, SYSTEM AND METHOD OF USE, which claims priority to U.S. Provisional Patent Application No. 63/364,255, filed May 5, 2022, and U.S. Provisional Patent Application No. 63/497,069, filed Apr. 19, 2023, both of which are incorporated herein by reference. Any and all applications for which a foreign or domestic priority claim is identified in the Application Data Sheet as filed with the present application are hereby incorporated by reference under 37 CFR 1.57.

### TECHNICAL FIELD

Embodiments of the present application relate to a surgical device, system and method of use and, more particularly, to a multi-functional, modular cautery device, system, and method of use.

### BACKGROUND

Endoscopic, minimally invasive, surgery relies on instrumentation for achieving hemostasis and surgical outcomes comparable to traditional open surgery techniques via comparatively small corridors, or ports (e.g., nostrils or keyholes) within a patient. Conventionally used bipolar cautery forceps face difficulties for use through the smaller corridors of this minimally invasive surgery. Presently used bipolar cauterization instruments suffer from limited mobility and visualization within the smaller corridors of minimally-invasive surgery and are difficult to use due to the relatively poor depth perception and stereoscopic vision offered within those corridors.

### SUMMARY

Minimally invasive and robotic surgical platforms are emerging rapidly across the spectrum of surgical disciplines. Commensurate with this are the development of novel surgical instrument arrays that attempt to integrate numerous surgical instruments into a given system, device, end effector, or platform. U.S. Pat. Nos. 9,033,974, 9,433,458 and 10,548,656, the entireties of which are hereby incorporated by reference, describe the use of a multi-functional surgical system that relies on standard surgical instruments including suction devices, microscissors, micrograsping forceps, dissectors, and curettes, among others, and augments each to provide bipolar or sesquipolar electrocautery between any two devices integrated into a standard RF surgical generator circuit. Embodiments of the present application are directed to improvements that may be applied to these and other devices, systems and methods, including other surgical platforms, to facilitate intraoperative efficiency when using numerous surgical instruments.

In some embodiments, a surgical cautery device, system, and method of use are herein described. The device involves a modified method of applying bipolar and/or sesquipolar electrocautery to target tissue via a pair of instruments that retain other primary surgical functions. The device may include a first and second element. The second element may be independently positionable with respect to the first element. The first and second elements include a surgical component and may be capable of forming an electrical circuit. The surgical component may be made from an electrically conductive material, such as stainless steel. Exemplary surgical components include a cutting tool, rotary blade, grasper tool, micro-grasping forceps tool, ring curette, dissector or micro-dissector, drill, ultrasonic tissue aspirator, micro-scissors tool, and a suction cannula, although a wide variety of insulated surgical instruments may be incorporated into this system. The surgical components are interchangeable, and can therefore be used in any combination to provide cautery application and increase efficiency of the operation. For example, when one surgical component is a suction cannula, it may be interchangeable with a cutting tool, a rotary blade, a grasper tool, a microscissors tool, a micro-grasping forceps tool, a dissector, a micro-dissector, or another suction cannula.

In many instances, the first and second elements are configured to contact a target tissue of a patient and, upon completion of the electrical circuit, deliver electrical energy to the target tissue. Often times, the delivery of the electrical energy to the target tissue acts to cauterize the target tissue.

Often times, a tip of the first and second elements may be electrically conductive while a portion of the first and second elements are electrically insulated from the tip. The first element and the second element may approach the target tissue through, for example, a conventional type of surgical opening, a single port (e.g., an endoscopic or microsurgery port), or a plurality of separate ports in the patient and may be configured to be manipulated by, for example, by a human surgeon and/or a robot.

Another exemplary device includes an electrically conductive wire that is electrically connected to an electrically insulated element. The electrically insulated element may include an electrically conductive surgical component. The surgical component may be capable of delivering electrical energy to a target tissue of a patient via the electrically conductive wirc.

Exemplary systems consistent with embodiments of the present application include a source of electrical energy electrically coupled to the first and second elements. The second element may be independently positionable with respect to the first element. The first and second elements may have a surgical component and may be capable of forming an electrical circuit and delivering electrical energy from the source to a target tissue of a patient upon completion of the electrical circuit. The systems may deliver, for example, cautery, sesquipolar cautery, and/or bipolar cautery.

Additional embodiments of the present application adapt one or more of the surgical instruments described above or elsewhere in this application to provide for one or more “smart tools.”

In some embodiments, an electrosurgical system for use in manipulating and cauterizing target tissue, can include: a plurality of surgical instruments, wherein cach of the plurality of surgical instruments can include: a sensor configured to detect when the surgical instrument is in use; an accelerometer; and a surgical tool at a working end of the surgical instrument, wherein the surgical tool can be configured to deliver electrical energy to a target tissue site and to perform an additional function other than delivering the electrical energy to the target tissue site; and a source of electrical energy configured to be electrically coupled to the plurality of surgical instruments, the source of electrical energy may include a controller, wherein the controller can be configured to: determine a distance between the working ends of at least two surgical instruments based on measurements from the accelerometers; calculate an impedance of the system based on the surgical tool and the target tissue; determine a proper current to deliver to the two working ends based on the impedance calculation; and deliver the determined proper current to the two working ends when the target tissue is positioned between the two working ends.

In some embodiments, the plurality of surgical instruments can include at least two surgical instruments each including a different type of surgical tool.

In some embodiments, the plurality of surgical instruments includes six surgical instruments each including a different type of surgical tool.

In some embodiments, the plurality of surgical instruments can include between two surgical instruments and six surgical instruments.

In some embodiments, the determined proper current can be based on a predetermined maximal voltage.

In some embodiments, the electrosurgical system, can include an adaptor, wherein the adaptor can be configured to couple the plurality of surgical instruments to the source of electrical energy.

In some embodiments, the adaptor can include the controller.

In some embodiments, the electrosurgical system, can include a first adaptor and a second adaptor, wherein the first adaptor can be configured to couple a first plurality of surgical instruments to the source of electrical energy and the second adaptor can be configured to couple a second plurality of surgical instruments to the source of electrical energy.

In some embodiments, the first plurality of surgical instruments can include surgical instruments configured for use with a right hand and the second plurality of surgical instruments can include surgical instruments configured for use with a left hand.

In some embodiments, the first adaptor and the second adaptor can each include a plurality of first halves of an induction charger, and each of the plurality of surgical instruments can include a battery and a second half of an induction charger, and each of the plurality of surgical instruments can wirelessly communicate with the first adaptor and the second adaptor, and when the plurality of the first halves of the induction charger are coupled to at least one of the second halves of the induction charger, the source of electrical energy can charge at least one of the batteries.

In some embodiments, each of the plurality of instruments can include a light source configured to light the tissue.

In some embodiments, the light source can be a light emitting diode.

In some embodiments, the light source can be configured to light an optical florescence agent or surgical dye.

In some embodiments, the controller can be configured to deliver the determined proper current to the plurality of instruments in use when the distance between the working ends of the plurality of instruments is less than a cutoff distance.

In some embodiments, the cutoff distance can be 5 mm.

In some embodiments, the controller can be configured to determine a type of the target tissue based on an impedance of the target tissue.

In some embodiments, the controller can use Electrochemical Impedance Spectroscopy (EIS) to determine the type of the target tissue.

In some embodiments, the controller can include an artificial intelligence and/or machine learning algorithm.

In some embodiments, the plurality of surgical instruments can each be insulated.

In some embodiments, the sensor can be a pressure sensor.

In some embodiments, the plurality of surgical instruments can each include a different surgical tool, and wherein the controller can be configured to automatically detect each different surgical tool.

In some embodiments, the source of electrical energy can include a memory, and the controller can determine a proper current to deliver to the two working ends based on information stored in the memory.

In some embodiments, an electrosurgical system for use in manipulating and cauterizing target tissue can include: a plurality of surgical instruments, wherein each of the plurality of surgical instruments can include: at least one sensor; and a surgical tool at a working end of the surgical instrument, wherein the surgical tool can be configured to deliver electrical energy to a target tissue site and to perform an additional function other than delivering the electrical energy to the target tissue site; and a source of electrical energy configured to be electrically coupled to the plurality of surgical instruments, the source of electrical energy including a controller, wherein the controller is configured to determine a desired operating condition based on a size, a shape or a type of the surgical instruments coupled to the source of electrical energy and/or based on information received from the at least one sensor.

In some embodiments, the at least one sensor can include an accelerometer and/or a gyroscope.

In some embodiments, the at least one sensor can be configured to detect when the surgical instrument is in use.

In some embodiments, the electrosurgical system can include an adaptor, wherein the adaptor can be configured to couple the plurality of surgical instruments to the source of electrical energy.

In some embodiments, the adaptor can include the controller.

### BRIEF DESCRIPTION OF THE DRAWINGS

Embodiments of the present application are illustrated by way of example, and not limitation, in the figures of the accompanying drawings in which:

FIG. 1 depicts an exemplary surgical system;

FIGS. 2A-2D depict exemplary first and/or second elements;

FIG. 2E depicts various exemplary surgical components;

FIGS. 3A-3C depict various exemplary sets of surgical components, consistent with embodiments of the present application;

FIG. 4 illustrates an exemplary use of first and second elements; and

FIG. 5 illustrates an exemplary surgical system incorporating a pair of adapters and a plurality of surgical instruments.

FIG. 6 illustrates an exemplary surgical system with elements categorized by handedness.

FIG. 7 illustrates a set of surgical components for use with embodiments of the present application.

### DETAILED DESCRIPTION

### Certain Embodiments of Electrosurgical Devices, Systems and Methods

Electrosurgical devices apply a high-frequency electric current to biological target tissue to cut, coagulate, or desiccate the target tissue or at least a portion of the target tissue. Electrosurgical devices use a generator (e.g., power supply or waveform generator) and a hand piece including one or several electrodes. Electrosurgery techniques are used in, for example, dermatological, gynecological, cardiac, plastic, ocular, spine, car, nose, and throat (ENT), maxillofacial, orthopedic, urological, neuro-and general surgical procedures as well as certain dental procedures.

One of the benefits of modern endoscopic surgery is the ability to work through two or more ports, via a bimanual and/or robotic approach. Rather than constrain the size and mobility of a cautery device to one port, one embodiment of the current surgical system proposes a novel electrocautery technique, in which two separate “electrodes” of the system are also independently insulated modular devices with their own functional purpose (e.g., micro-grasping forceps, suction cannula, micro-scissors, dissectors, micro-dissectors, etc.). These dually or multiply functioning components of the cautery system can manipulate target tissue with much greater mobility and visualization, independently transmit opposing current from one electrode to another in order to achieve a sesquipolar or bipolar cautery effect (depending on, for example, the size and surface area of the conducting electrode surfaces) from one electrode to the other, and/or retrieve data or information from the two tips such as electrical impedance of the target tissue. Rather than functionally diverge near the tip of the forceps, as current models for endoscopic bipolar forceps propose, certain implementations of the present application have two separate electrodes with dual function as one or more other surgical devices. The two electrodes diverge outside of the patient rather than within the surgical cavity, and are connected to each other and a power supply via wiring in order to appropriately transmit opposing high-frequency current to contacted target tissue. Each functional electrode/element of the electrocautery device may be insulated with respect to the surgical component, so that current will only be transmitted selectively from one surgical component to the other. The modular devices can be connected and disconnected to, for example, standard wires used with power supplies, such as bipolar electro cautery generators, and may be used in various combinations (e.g., suction cannula and micro-scissors or micro-grasping forceps and micro-scissors). Current may be activated via any conventionally available means, such as with a foot pedal in a manner similar to existing bipolar devices.

Embodiments of the present application provide increased mobility and visualization in cauterizing the surgical target when compared with conventional techniques, by, for example, allowing two or more elements with surgical components to approach target tissue from different depths, angles, and/or ports. Each surgical component may have independent, interchangeable, and/or functional properties (i.e., cutting, grasping, dissection, sucking, probing, etc.), thus allowing a surgeon to manipulate delicate surgical target tissue as it is cauterized in an efficient manner. In addition, according to the present invention, the size of a surgical opening within a patient (i.e., port) need only accommodate one surgical component, which, in many cases, is smaller than traditionally used cauterizing forceps, and may be as small as 1 mm or less in some examples.

Embodiments of the present application further allow a surgeon to perform surgical operations and cauterize with the same surgical components, thereby reducing the need to remove surgical devices from the patient and subsequently insert a separate cauterization device. Thus, utilization of these embodiments increases surgical efficiency and potentially reduces the risk of infection or damage to surrounding anatomical structures that may be caused by repeatedly removing and inserting devices.

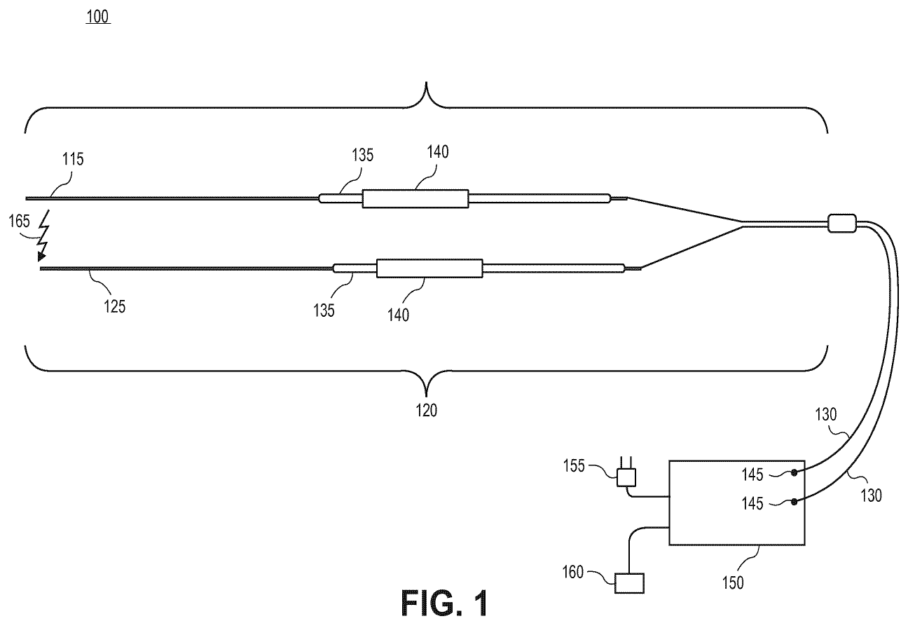

Embodiments of the present application are more particularly described with regard to the exemplary embodiments depicted in the figures that accompany the instant patent application. For example, FIG. 1 depicts an exemplary surgical system 100 consistent with some embodiments. Surgical system 100 may include a power supply 150, a power cord 155, and an activation device 160. Power supply 150 may be coupled to a first element 110 and a second element 120 via an electrical connector 145 (e.g., banana clip) electrically coupled to an electrically conductive wire 130. Power supply 150 may be any device capable of supplying electrical power, or current, to first and second elements 110 and 120 upon user selection of activation device 160. Activation device 160 may be any conventionally available means for initiating the delivery of electricity to first element 110 and/or second element 120 including, but not limited to, a foot pedal, a button, or a dial. In some embodiments, an amount of power delivered to first and/or second elements 110 and 120 may be controlled by manipulation of activation device 160 (e.g., twisting a dial) in order to deliver a maximum level of power, or a fraction thereof, to first and/or second elements 110 and 120.

First and second elements 110 and 120 may be configured to deliver electrical energy 165 from power supply 150 to a contacted, or target, portion of tissue within a patient via surgical components 115 and/125. Exemplary target tissue includes a small blood vessel in need of cauterization, tumor, or other undesirable tissue to be removed from the patient. First and second elements 110 and 120 may be configured to be manipulated by a human surgeon and/or a robot and, on some occasions, may be configured to be used in microscopic or endoscopic single or multiple port surgery. In some embodiments, a portion of first and second elements 110 and 120, with the exception of a first and second surgical components 115 and 125, respectively, may be covered in electrical insulation 135 or may be otherwise insulated. In this way, only surgical components 115 and/or 125 may deliver electrical energy from power supply 150 to contacted tissue. Electrical insulation 135 may be any appropriate electrically insulating material including, but not limited to, plastic, vinyl, epoxy, parylene, or ceramic and may enable a surgeon to grasp and/or hold first and second elements 110 and 120 via, for example, graspers 140. First and/or second elements 110 and 120 as well as surgical components 115 and/or 125 may be disposable (i.e., one time use), or reusable (i.e., capable of being used multiple times).

On some occasions, first and second surgical components 115 and 125 may be similarly configured to one another with regard to shape and size and, in some instances, may comprise a matched pair of components. On other occasions, first surgical component 115 may be configured to perform a first function in addition to the conduction of electricity and second surgical component 125 may be configured to perform a second function in addition to the conduction of electricity. For example, first surgical component 115 may be configured to be operable by a robot while second surgical component 125 may be configured to be operable by a human surgeon. Additionally, one or both surgical components 115 and/or 125 may include one or more controls (not shown) that enable a manipulator of the surgical component (e.g., human surgeon or robot) to control the operation of the surgical component.

First and second elements 110 and 120 and/or first and second surgical components 115 and 125 may configured to be independently positionable by a human surgeon and/or a robot. In this way movement of, for example, first element 110 does not impact the position of second element 120. Likewise, on some occasions, movement of first surgical component 115 may not impact the position or functioning of second surgical component 125. In this manner, first and second elements 110 and 120 and/or first and second surgical components 115 and 125 may be moved independently within a patient and/or prior to entry into a patient to, for example, contact target tissue from different angles or enter different ports within a patient and/or perform different functions (in addition to the delivery of electricity) within the patient with regard to the target tissue.

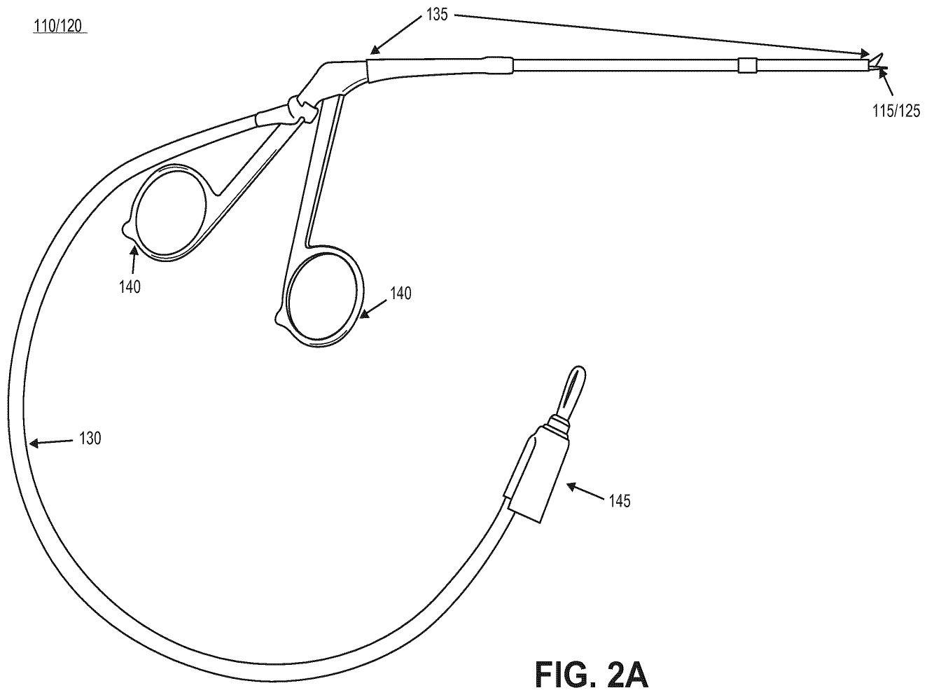

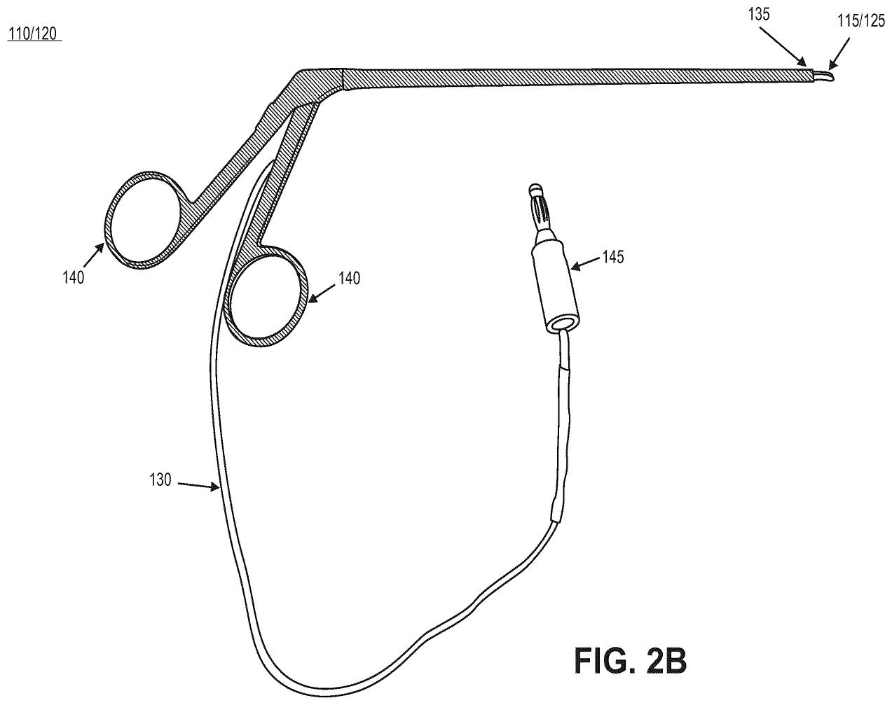

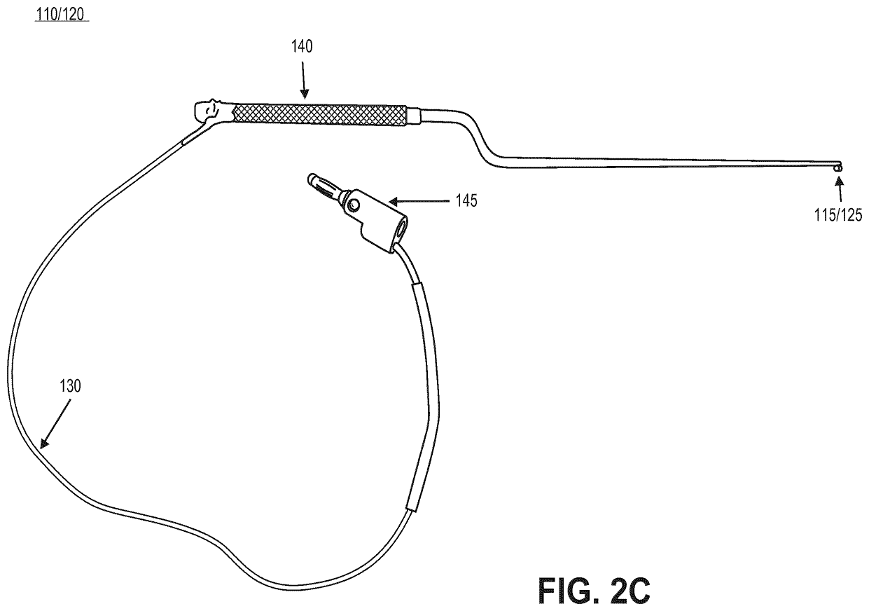

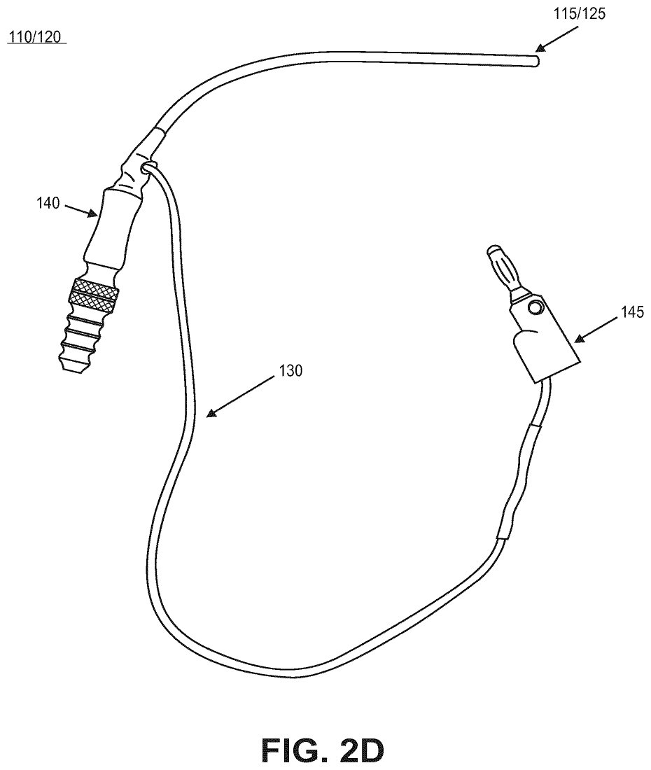

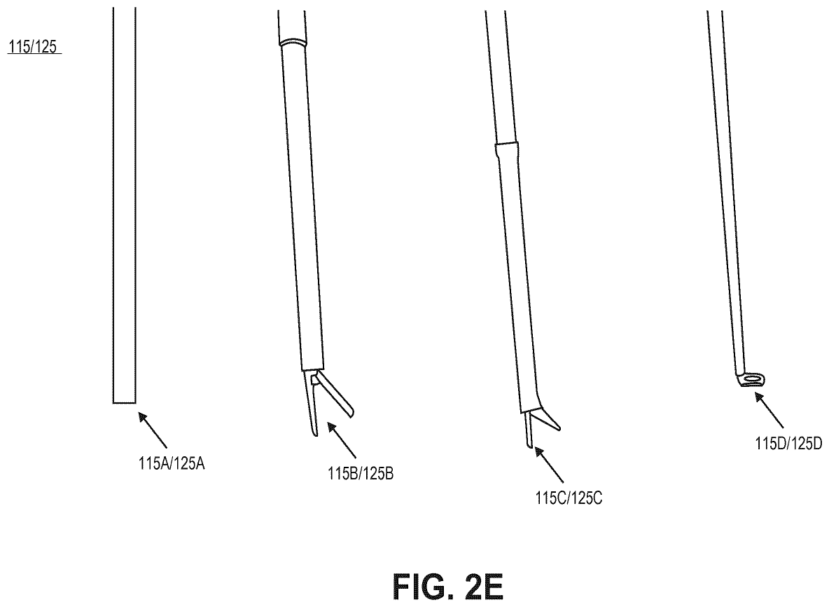

In some embodiments, first and second elements 110 and 120 may be interchangeable with other elements via any known method. For example, first and/or second element 110 and/or 120 may be interchangeable at power supply 150 via extraction of electrical connector 145 coupled to first or second element 110 or 120 from power supply 150 and insertion of another electrical connector compatible with power supply 150 (not shown) electrically coupled to another element (not shown) into power supply 150. In this way, for example, micro-scissors element 110/120 as depicted in FIG. 2A (described below) may be interchanged with suction cannula element 110/120 as depicted in FIG. 2D (described below). Additionally or alternatively, surgical components 115 and/or 125 may be interchangeable with other surgical components via any conventionally available means, including, but not limited to, unscrewing or otherwise decoupling surgical component 115 and/or 125 from first and/or second elements 110 and 120. For example, a surgical component 115 or 125 may be removed from element 110 or 120, respectively, and another surgical element may be attached to the first or second element 110 or 120.

FIGS. 2A-2D depict exemplary first and/or second elements 110/120. In FIG. 2A, first and/or second element 110/120 is configured as a micro-scissors tool, wherein graspers 140 are embodied as scissor handles, the shaft of the micro-scissors tool is encased in insulation 135 and surgical component 115/125 is an electrically conductive set of micro-scissors. In FIG. 2B, first and/or second element 110/120 is also configured as a micro-scissors tool, wherein the entire first and/or second element 110/120, with the exception of surgical component 115/125, is covered with insulation 135. In FIG. 2C, first and/or second element 110/120 is configured as a probe, wherein surgical component 115/125 is a surgical probe. The first and/or second element 110/120 of FIG. 2C may also include a handle 170. In FIG. 2D, first and/or second element 110/120 is configured as a suction tool, wherein surgical component 115/125 is a suction cannula. FIG. 2E depicts various exemplary surgical components 115/125, wherein surgical component 115A/125A is a suction cannula, surgical component 115B/125B is a grasper, surgical component 115C/125C is a set of micro-scissors, and surgical component 115D/125D is a probe.

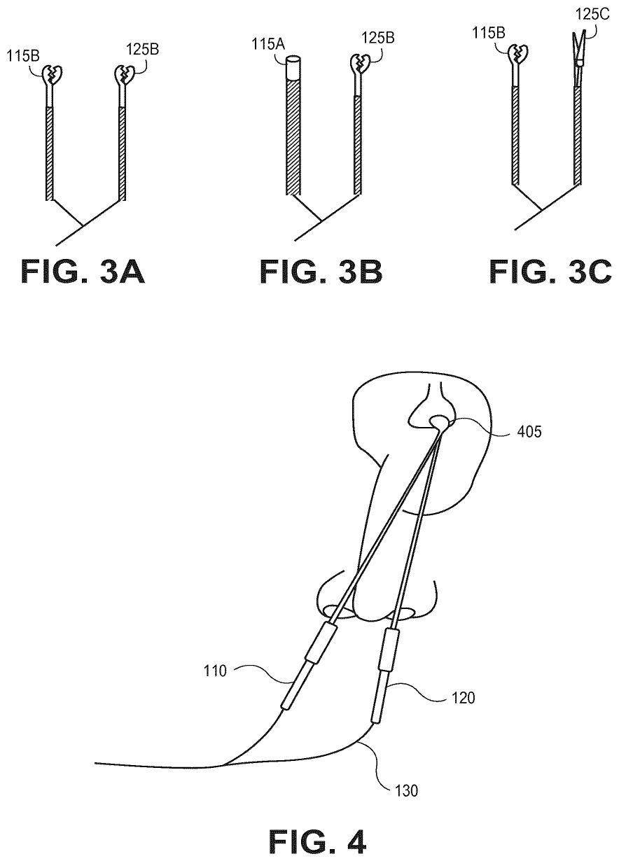

In some embodiments, first and second surgical components may be similar to, or different from, one another. For example, FIGS. 3A-3C depict various exemplary sets of surgical components 115 and 125 as provided by various embodiments of the present invention. As depicted in FIG. 3A, first and second surgical components 115B and 125B are configured as grasping elements that enable a surgeon to grasp and manipulate target tissue as well as cauterize the target tissue. As depicted in FIGS. 3B and 3C, surgical components 115 and 125 are configured differently from one another. In the embodiment depicted in FIG. 3B, surgical component 115A is configured as a suction device and surgical component 115B is configured as a grasping component. A surgeon utilizing first and second elements 110 and 120 of this embodiment would thus be enabled to grasp target tissue, suck material (e.g., blood, bone, and/or target tissue) from the patient, and cauterize target tissue while, for example, suctioning smoke resulting from cauterization to improve visualization. In the embodiment depicted in FIG. 3C, surgical component 115B is configured as a grasping tool and surgical component 115C is configured as a micro-scissors tool. A surgeon utilizing first and second elements 110 and 120 of this embodiment would thus be enabled to grasp, cut, and cauterize target tissue without requiring removal or insertion of any additional devices.

FIG. 4 illustrates an exemplary use of first and second elements 110 and 120 following insertion into two ports of a patient to contact target tissue 405. In this embodiment, first element 110 is inserted into a first port within the right nostril of a patient and second element 120 is inserted into a second port within the left nostril of the patient. In this way, first and second elements may approach target tissue 405 from different angles and may move independently of one another. Following insertion of first and second elements 110 and 120 into the first and second ports within the patient, the delivery of electricity may be initiated via user selection of activation device 160 of power supply 150 thereby forming an electrical circuit. Following activation, electrical power may be delivered to first and/or second elements 110 and/or 120 and, upon contact of surgical components 115 and 125 with target tissue, electrical energy 165 may be delivered to the target tissue, thereby cauterizing the target tissue. The same application could be used for multi-port surgery in the abdomen, thorax, or any other surgical site where one or multiple access ports or corridors are utilized.

### Further Improvements to Facilitate Intraoperative Efficiency

The devices, systems and methods described above may further comprise one or more improvements to facilitate surgical efficiency and ease of use in the operating environment, as described further below. Although these further improvements are described with reference to the devices, systems and methods of FIGS. 1-4, these improvements may also be applied to other devices, systems and methods as well.

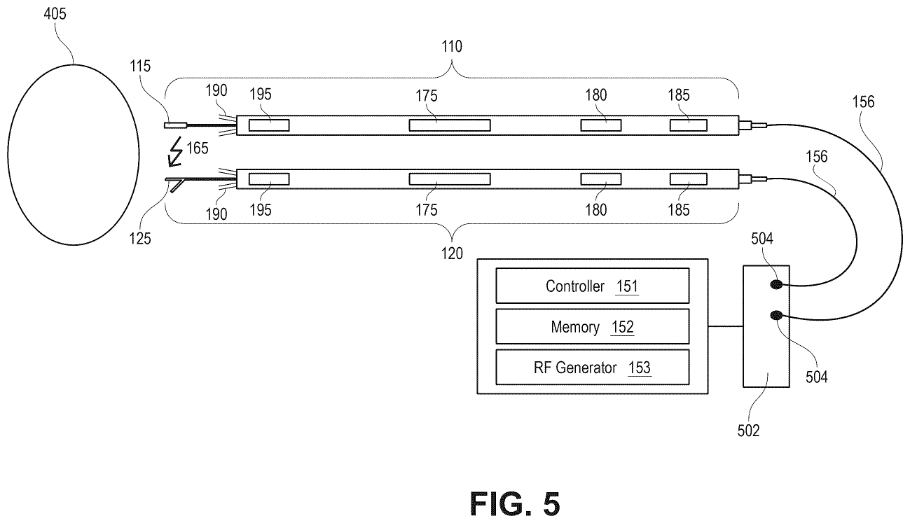

As shown in FIG. 5, in some embodiments, a surgical instrument, such as the first element 110 and/or the second element 120, may include at least one sensor 175. The at least one sensor 175 may include a pressure sensor, capacitive touch sensor, or other sensor configured to detect if the human surgeon or robot is holding the first element 110 and/or the second element 120. The at least one sensor 175 may be coupled to graspers 140 or electrical insulation 135, or a handle or other location on the surgical instrument.

In some embodiments, the system 100 may include a hub herein referred to as the power supply 150, though it will be appreciated that the power supply 150 or hub may comprise further components and features beyond only supplying power. In some embodiments, the hub or the power supply 150 may include one or more processors or controllers 151. The at least one sensor 175 may be configured to communicate with the one or more processors or controllers 151 via a connection 156, e.g., a wired or wireless connection. The one or more processors or controllers 151 may control the flow of electrical energy 165 to first and second elements 110 and 120 and/or the surgical components 115 and 125 positioned at a working end of the first and second elements 110 and 120. For example, in one embodiment, power supply 150 may deliver electrical energy 165 to first and second elements 110 and 120 when the at least one sensor 175 detects the human surgeon or robot is holding and/or moving the first and/or second elements 110, 120. In another embodiment, upon user selection of activation device 160, the power supply 150 may only deliver electrical energy to the first and second elements 110 and 120 that the at least one sensor 175 detects are in use. In some embodiments, the power supply 150 may include an RF generator 153. The RF generator 153 may be configured to generate the electrical energy 165 or current delivered to the target tissuc 405.

In some embodiments, a surgical instrument, such as the first and second elements 110 and 120, may each also include at least one location or motion sensor 195, such as an accelerometer, gyroscope, inertial measurement unit (IMU), or other similar sensors. The location or motion sensor 195 may be configured to send element location or motion data to the one or more processors or controllers 151. The one or more processors or controllers 151 may receive the element location or motion data from the location or motion sensor 195 and determine a location or related information of the first and second elements 110 and 120 relative to each other. The one or more processors or controllers 151 may be able to use the element location data or related information to determine the distance between the surgical components 115 and 125. In some embodiments, one or more processor or controllers 151 of the power supply 150 may include a stereotactic navigation processor to determine the location of the first and second elements 110 and 120.

In some embodiments, the one or more processors or controllers 151 may communicate with the at least one location or motion sensor 195 to detect or determine if the human surgeon or robot is holding the first element 110 and/or the second element 120. In some embodiments, the one or more processor may communicate with the at least one location or motion sensor 195 to detect or determine which of the first element 110 and/or the second element 120 are in use by the human surgeon or robot.

In some embodiments, the one or more processors or controllers 151 may control an amount of the electrical energy 165 or current the power supply 150 delivers to the target tissue 405. The amount of the electrical energy 165 or current the power supply 150 delivers to the target tissue 405 may depend on the size, shape and/or type of surgical components 115 and 125 in use, an impedance of the target tissue 405, e.g., bone, neural, vessels, etc., and/or the distance between the surgical components 115 and 125. In some embodiments, the human surgeon may input the type of target tissue 405 via a user interface. In some embodiments, the one or more processors or controllers 151 may communicate with the surgical components 115 and 125 to determine the impedance of the target tissue 405 and/or the system 100. In these embodiments, the one or more processors or controllers 151 may use feedback from the surgical components 115 and 125 to calculate the impedance of the system 100. The system 100 may be configured to detect the type of the target tissue 405 based on the calculated impedance of the target tissue 405. The one or more processors or controllers 151 may dynamically adjust the amount of the electrical energy 165, voltage, and/or current supplied to the surgical components 115 and 125 in real time or substantially real time based on the calculated impedance of the target tissue 405.

In some embodiments, the one or more processors or controllers 151 may adjust the amount of current supplied to the surgical components 115 and 125 in real time or about real time. The one or more processors or controllers 151 may use a feedback loop from the surgical components 115 and 125 to determine the amount of current to deliver to the target tissue 405. The one or more processors or controllers 151 may shut off the current when the system detects an impedance associated with a certain tissue. In some embodiments, the certain tissue may be bone, a tumor, for example, a brain tumor, or nerve. In some embodiments, the one or more processors or controllers 151 may comprise an algorithm configured to control the amount of current delivered to the tissue based on a detected impedance of the tissue.

In some embodiments, the algorithm may include artificial intelligence (AI) and/or machine learning (ML). The algorithm may use AI and/or ML to determine the type of the target tissue 405 (i.e., provide tissue diagnostics) based on the calculated impedance of the target tissue 405. For example, the target tissue may include an abnormal impedance level, and the algorithm may determine the type of the target tissue 405 is cancerous tissue. In some embodiments, the algorithm may determine the type of target tissue 405 in real time or substantially real time.

In some embodiments, the one or more processors or controllers 151 and/or the algorithm may be configured to use Electrochemical Impendence Spectroscopy (EIS) to calculate the impedance of the target tissue 405 and/or determine the type of the target tissue 405. In some embodiments, the electrical energy 165 or current may include a direct current (DC). In some embodiments, the electrical energy 165 or current may include an alternating current (AC). The one or more processors or controllers 151 and/or the algorithm may apply the AC to the target tissue 405 over a period of time. The one or more processors or controllers 151 and/or the algorithm may modify, vary, or adjust a frequency of the AC over the period of time. In some embodiments, the one or more processors or controllers 151 and/or the algorithm may apply a high frequency AC to the target tissue 405 and reduce the frequency of the AC over the period of time. In some embodiments, the one or more processors or controllers 151 and/or the algorithm may apply a low frequency AC to the target tissue 405 and increase the frequency of the AC over the period of time. In some embodiments, the frequency of the AC may include a frequency of about 1 Hz, about 10 Hz, about 100 Hz, about 1 kHz, about 10 kHz, about 20 kHz, about 50 kHz, about 100 kHz, about 150 kHz, about 200 kHz, about 300 kHz, about 400 kHz, about 500 kHz, about 600 kHz, about 700 kHz, about 800 kHz, about 900 kHz, about 1 MHz, about 10 MHz, about 20 MHZ, about 30 MHz, about 40 MHz, about 50 MHz, about 60 MHz, about 70 MHz, about 80 MHz, about 90 MHz, about 100 MHZ, about 500 MHz, about 1,000 MHz, and/or any value between the aforementioned values. In some embodiments, the frequency if the AC may include a frequency greater than 1,000 MHz. In some embodiments, the frequency of the AC may include a frequency less than 1 Hz. In some embodiments, the frequency of the AC may include a frequency between about 1 kHz and about 500 kHz. In some embodiments, the frequency of the AC may include a frequency between about 50 kHz and about 150 kHz.

In some embodiments, the one or more processor or controllers 151 and/or the algorithm may determine or measure a magnitude and/or a phase angle of the impedance or a spectral readout. In some embodiments, the one or more processor or controllers 151 and/or the algorithm may automatically determine the type of the target tissue 405 based on the magnitude and/or the phase angle of the impedance or the spectral readout.

In some embodiments, the algorithm may use AI and/or ML to automatically determine the size, shape and/or type of surgical components 115 and 125 in use, a distance between the surgical components 115 and 125, and/or an impendence of the system 100. The algorithm may determine the size, shape and/or type of surgical components 115 and 125 in use, a distance between the surgical components 115 and 125, and/or an impendence of the system 100 in real time or substantially real time.

In some embodiments, the algorithm may use AI and/or ML to adjust and/or modulate the amount of the electrical energy 165, voltage, and/or current supplied to the surgical components 115 and 125 and/or the target tissue 405. The algorithm may adjust and/or modulate the amount of the electrical energy 165, voltage, and/or current supplied to the surgical components 115 and 125 and/or the target tissue 405 in real time or substantially real time. In some embodiments, the algorithm may adjust and/or modulate the amount of electrical energy 165, voltage, and/or current supplied to the surgical components 115 and 125 and/or the target tissue 405 based on an amount of time the electrical energy 165, voltage, and/or current has been supplied to the surgical components 115 and 125 and/or the target tissue 405 (i.e., a firing duration).

In some embodiments, the human surgeon may input, into the power supply 150, the type of surgical components 115 and 125 currently in use. In other embodiments, the power supply 150 may automatically detect and determine the type of surgical components currently in use. In these embodiments, the surgical components 115 and 125 may comprise a memory 180 configured to store information associated with the type of surgical component. In some embodiments, the information may include the type of surgical component, dimensions of the surgical component, or any other information associated with the surgical components 115 and 125.

The power supply 150 may have a predetermined impedance associated with different sizes and/or shapes of different types of surgical components 115 and 125 stored in a memory 152. In some embodiments, the memory 180 of the first and second elements 110 and 120 may be configured to store a predetermined impedance associated with a size, shape and/or type of surgical components 115 and 125 respectively. The first and second elements 110 and 120 may be configured to send or transmit the predetermined impedance associated with the size, shape and/or type of the surgical components 115 and 125 to the one or more processors or controllers 151 of the power supply 150 when the at least one sensor 175 detects that the first and second elements 110 and 120 are in use. In some embodiments, the power supply 150 may detect the type and/or size and shape of each of the surgical components 115 and 125 connected to the system 100, whether or not the surgical components 115 and 125 are currently in use. In some embodiments, a human surgeon may input a predetermined maximal voltage or into the power supply 150 via a user interface. The one or more processors or controllers 151 may determine a proper current to deliver to the target tissue 405 based on the predetermined maximal voltage, and the impedance of the system 100. In some embodiments, the one or more processors or controllers 151 may send current to the surgical components 115 and 125 in use.

In some embodiments, the one or more processors or controllers 151 may compare information received from the at least one location or motion sensor 195 of the first and second elements 110 and 120, and/or the surgical components 115 and 125 to determine the impedance of the target tissue 405 or the system 100, the type of the target tissue 405, and/or the distance between the surgical components 115 and 125. In some embodiments, the one or more processors or controllers 151 may compare the impedance of the target tissue 405 or the system 100, the type of the target tissue 405, and/or the distance between the surgical components 115 and 125 to data or information stored in the memory 180 to determine and/or set thresholds or limits for the amount of electrical energy 165, voltage, and/or current that the power supply 150 may deliver to the surgical components 115 and 125 and/or the target tissue 405.

In some embodiments, the one or more processors or controllers 151 may stop the flow of electrical energy 165 or the current when the distance between the surgical components 115 and 125 is longer than a predetermined cutoff distance. In some embodiments, the cutoff distance may include a distance of about 1 mm, about 2 mm, about 3 mm, about 4 mm, about 5 mm, about 10 mm, about 15 mm, about 20 mm, and/or any other value between the aforementioned values.

In some embodiments, as shown in FIG. 5, the system 100 may include at least one adaptor 502. The at least one adaptor 502 may be coupled to the power supply 150 and may include one or more ports 504. The one or more ports 504 may be designed to electrically couple the first and/or second elements 110 and 120 to the at least one adaptor 502 via the connection 156. The at least one adaptor 502 may be configured to electrically couple any number of elements to the power supply 150. The adaptor 502 may include the one or more processors or controllers 151 configured to control whether first and second elements 110 and 120 deliver electrical energy 165.

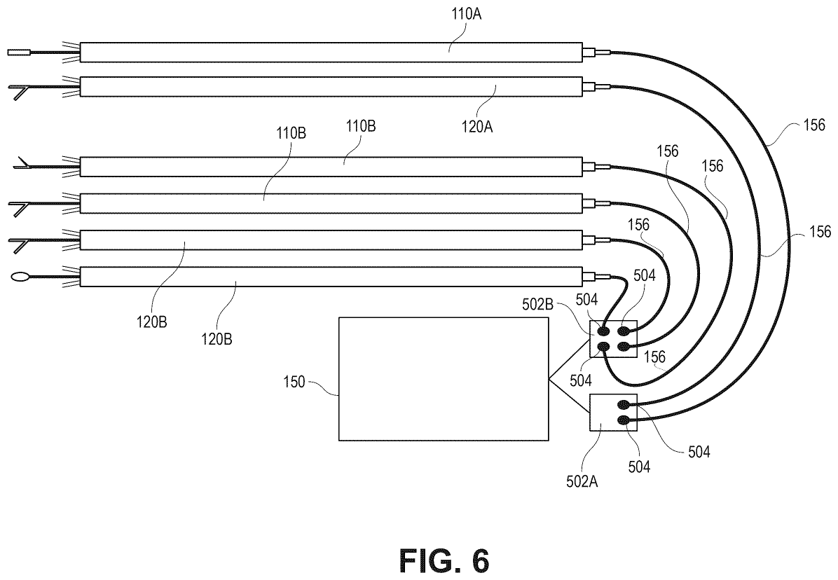

In some embodiments, the system 100 includes one or more adaptors 502 configured to electrically couple the first and second element 110 and 120 to the power supply 150. In some embodiments, as shown in FIG. 6, a first adaptor 502A may be configured to receive elements the human surgeon uses with a right hand, and a second adaptor 502B may be configured to receive elements the human surgeon uses with a left hand. The first adaptor 502A may be configured to act as an anode, and the second adaptor 502B may be configured to act as a cathode, or vise-a-versa. Having a separate anode and cathode allows the system 100 to transfer energy between two separate surgical components in unison. In some embodiments, one adaptor 502 may be configured to receive both the elements the human surgeon uses with the right hand and the elements the human surgeon uses with the left hand. In some embodiments, the system 100 may automatically detect which hand the surgeon uses elements with or the surgeon may input into the at least one adaptor 502 and/or the power supply 150 what hand each element will be used with based on what surgery is performed.

In some embodiments, the one or more adaptors 502 may include one or more induction coils. The first and second elements 110 and 120 may include one or more induction coils and one or more batteries 185. In some embodiments, the one or more induction coils of the one or more adaptors 502 may include first halves of the induction charger, and each of the one or more induction coils of the first and second elements 110 and 120 may include second halves of an induction charger. Each of the one or more induction coils of the one or more adaptors 502 may include a first half of the induction charger, and each of the one or more induction coils of the first and second elements 110 and 120 may include a second half of the induction charger. In these embodiments, the one or more induction coils of the one or more adaptors 502 may charge the one or more batteries 185 of the first and second elements 110 and 120. In these embodiments, the first and second elements 110 and 120 may include a transceiver and the one or more adaptors 502 may include a transceiver. The connection 156 between first and second elements 110 and 120, and the one or more adaptors 502 may include a wireless connection between the transceiver of the first and second elements 110 and 120 and the transceiver of the one or more adaptors 502 so the first and second elements 110 and 120 may communicate with the one or more adaptors 502 wirelessly via the transceiver.

In some embodiments, the one or more adaptors 502 may comprise the one or more processors or controllers 151, the RF generator 153, and/or a suction device and may perform any function the power supply 150 performs above.

In some embodiments, the one or more adaptors 502 may be used to retrofit the system 100 to an existing power supply 150 that may not include features of system 100.

In some embodiments, the first and second elements 110 and 120 may include one or more light sources or lights 190. The one or more lights 190 may be configured to light the first and second surgical components 115 and 125 to improve visibility for the human surgeon when the human surgeon is performing surgery. In some embodiments, the one or more lights 190 may be configured to generate a wavelength of light. The wavelength of light may include a wavelength of white light. In some embodiments, the wavelength of light generated by the one or more lights 190 may be selected based on an optical fluorescence agent or surgical dye used during surgery. For example, the optical fluorescence agent or surgical dye may include fluorescein, indocyanine green, 5-ALA/protoporphyrin IX, and/or any other optical fluorescence agent or surgical dye. The wavelength of light generated by the one or more lights 190 may light or improve visibility of the optical fluorescence agent or surgical dye for the human surgeon when the human surgeon is performing surgery.

In some embodiments, the first and second elements 110 and 120 may comprise ultrasonic aspirator handpieces. In some embodiments, at least one of the first and second elements 110 and 120 may comprise a suction or irrigation device. In some embodiments, at least one of the first and second elements 110 and 120 may comprise a side-cutting mechanical aspiration device.

In some embodiments, the system 100 may use machine learning and/or artificial intelligence to learn the human surgeon's preferences over time. The human surgeon's preferences may include combinations of surgical components used together, a hand the surgeon uses to hold certain surgical components, and/or a predetermined maximal voltage preference based on the surgical components used by the human surgeon.

It is to be appreciated that although the system 100 is described as having first and second elements 110 and 120, the system may include more elements. In some embodiments, the system 100 may include three elements, four elements, five elements, six elements, or more. In some embodiments, the system 100 may include between two and six elements. In some embodiments, the system 100 may include particular set of elements configured for particular operations or surgical subspecialties. The set of elements typically used for the particular operations or surgical subspecialties. For example, a first set of elements may include elements typically used for a first operation, and a second set of elements may include elements typically used for a second operation.

As shown in FIG. 5, in one embodiment, first element 110 may include live suction, and second element 120 may include live scissors, live graspers or live dissectors. In this embodiment, the one or more lights 190 of the first element 110 and/or the second element 120 may include a light emitting diode (LED) and/or LED illumination. First and second elements 110 and 120 may be used by the human surgeon or robot to manipulate a target lesion or target tissue 405. The system 100 may use co-navigation to determine the location of first and second elements 110 and 120.

As shown in FIG. 6 the system 100 may comprise first and second elements 110 and 120 for use with the right hand of the surgeon and the left hand of the surgeon. Although certain first and second elements 110 and 120 are shown in FIG. 6 any first and second elements 110 and 120 may be used with the right hand of the surgeon and the left hand of the surgeon. In some embodiments, a hand an element is used with may be the same hand throughout a surgery, or the hand the element is used with may change during the surgery.

In some embodiments, the first element 110 may comprise a suction tool and a grasper tool. The second element 120 may comprise scissors, a dissector, a grasper and a curette. In some embodiments, the system 100 may be customized, individualized or modular to allow the human surgeon to select which elements are used for a particular surgery.

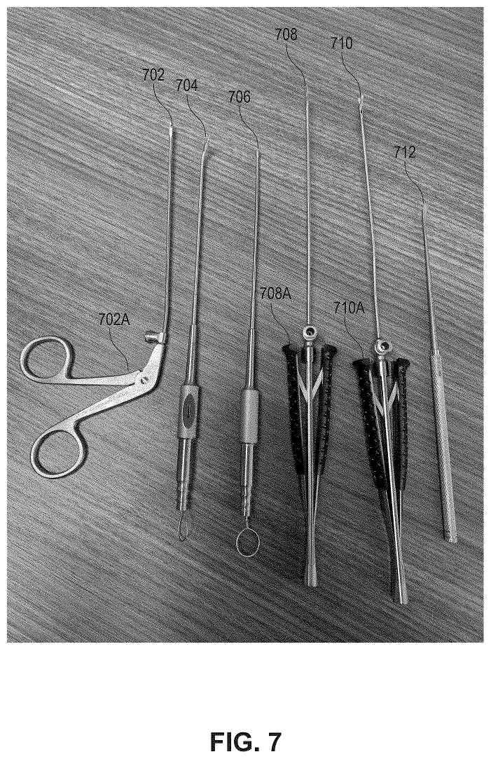

FIG. 7 shows examples of surgical components or surgical instruments that may be used with system 100. Examples of surgical components or surgical instruments that may be used with system 100 may include, first micro-graspers 702, a suction tool 704, a suction tool and dissector 706, microscissors 708, second micro-graspers 710, and/or a dissector 712. The first micro-graspers 702 may include a pistol grip 702A. The microscissors 708 and the second micro-graspers 710 may include a pinch grip 708A, 710A.

### EXAMPLE

In one example embodiment, the hub of a system as described above may include a processing unit with multiple ports for multiple surgical instruments that can be relayed to create a functional two-way circuit. The hub may include up to six ports or more. Multiple surgical instruments may be plugged into the system. The system may recognize each surgical instrument when plugged in. The hub may be an intermediary unit between multiple other inputs including an RF generator, a suction device, and a stereotactic navigation processor. The stereotactic navigation processor may determine the location of instrument tips based on actuators as well as a camera array that visualizes the instruments.

Surgeons typically use certain surgical instruments in their right or left hand depending on which hand is the surgeons dominant hand. For example, a right-handed surgeon may use a suction device in the surgeon's left hand, and scissors in the surgeon's right hand. The adaptor may take advantage of this preferential use of instruments to facilitate the completion of a circuit. The adaptor may include separate inputs for the anode and the cathode of the circuit. By having separate inputs, the adaptor may allow two separate instruments to be used in unison to transfer energy between two tips of the surgical instruments.

The surgical instruments may include an accelerometer and a pressure sensor on a handle. The processing unit may use the accelerometer and/or the pressure sensor to detect when the surgical instruments are in use by the surgeon. The processing unit may only activate instruments that are in use by the surgeon, as determined by the accelerometer and/or the pressure sensor. The surgical instruments not in use may be switched off by the processor. The circuit may use computational understanding of when the circuit is complete by using preapproved or preselected combinations of instruments to complete the circuit. The adaptor eliminates the step of the surgeon plugging in or unplugging instruments each time the surgeon decided to use different surgical instruments thereby improving workflow and efficiency.

The system may use electrode feedback from the tips of smart tools, including actuator location, impedance or resistance, and/or any physiological stimulation to calculate information including, the distance between the tips of two surgical instruments in use, the surface area, or size of the surgical instruments in use, and/or the impedance of intervening tissue. The system may use the information to determine a desired operating condition, implement safety mechanisms, set limits or thresholds for the amount of thermal energy delivered to the target tissue or tips of the surgical instruments, augment the amount of thermal energy delivered to the target tissue or allow tips of the surgical instruments to meet a via separate entry points. For example, the processing unit may use a measured impedance between two tips of the surgical instruments to deliver a predetermined current or maintain a predetermined maximal voltage between the two tips of the surgical instruments, or within the system, to reduce and/or safely limit the amount of thermal or electrical energy being delivered to intervening surgical tissue in the bipolar or sesquipolar system. The system may compare the distance between the tips of two surgical instruments in use, the surface area, or size of the surgical instruments in use, and/or the impedance of intervening tissue to information or data stored in a memory to determine the desired operating condition, implement safety mechanisms, set limits or thresholds for the amount of thermal energy delivered to the target tissue or tips of the surgical instruments, augment the amount of thermal energy delivered to the target tissue or allow tips of the surgical instruments to meet a via separate entry points.

The processing unit of the system may include artificial intelligence (AI) or machine learning (ML). The processing unit may automatically identify or determine the type of the intervening tissue based on the impedance of the intervening tissue. The processing unit may use Electrochemical Impedance Spectroscopy (EIS) to calculate the impedance of the intervening tissue and/or determine the type of the intervening tissue. The processing unit may determine a magnitude and/or a phase angle to the impedance of the intervening tissue, or a spectral readout to identify or determine the type of the intervening tissue. The processing unit may identify or determine the type of the intervening tissue in real time or substantially real time.

The subject technology is illustrated, for example, according to various aspects described above. The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that some or all steps, operations, or processes may be performed automatically, without the intervention of a user. Method claims may be provided to present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the appended claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Brief Description of the Drawings, and Claims of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in any claim. Rather, as the following claims s reflect, inventive subject matter lies in less than all features of a single disclosed configuration or operation. The following claims are hereby incorporated into the Detailed Description, with each claim standing on its own to represent separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An electrosurgical system for use in manipulating and cauterizing target tissue, comprising:
a plurality of surgical instruments, wherein each of the plurality of surgical instruments comprises:
a sensor configured to detect when the surgical instrument is in use;
an accelerometer; and
a surgical tool at a working end of the surgical instrument, wherein the surgical tool is configured to deliver electrical energy to a target tissue site and to perform an additional function other than delivering the electrical energy to the target tissue site; and
a source of electrical energy configured to be electrically coupled to the plurality of surgical instruments, the source of electrical energy comprising a controller, wherein the controller is configured to:
determine a distance between the working ends of at least two surgical instruments based on measurements from the accelerometers;
calculate an impedance of the system based on the surgical tool and the target tissue;
determine a proper current to deliver to the two working ends based on the impedance calculation; and
deliver the determined proper current to the two working ends when the target tissue is positioned between the two working ends.

2. The electrosurgical system of claim 1, wherein the plurality of surgical instruments comprises at least two surgical instruments each comprising a different type of surgical tool.

3. The electrosurgical system of claim 1, wherein the plurality of surgical instruments comprises six surgical instruments each comprising a different type of surgical tool.

4. The electrosurgical system of claim 1, wherein the determined proper current is based on a predetermined maximal voltage.

5. The electrosurgical system of claim 1, further comprising an adaptor, wherein the adaptor is configured to couple the plurality of surgical instruments to the source of electrical energy.

6. The electrosurgical system of claim 5, wherein the adaptor comprises the controller.

7. The electrosurgical system of claim 1, further comprising a first adaptor and a second adaptor, wherein the first adaptor is configured to couple a first plurality of surgical instruments to the source of electrical energy and the second adaptor is configured to couple a second plurality of surgical instruments to the source of electrical energy.

8. The electrosurgical system of claim 7, wherein the first adaptor and the second adaptor each comprises a plurality of first halves of an induction charger, and each of the plurality of surgical instruments comprise a battery and a second half of an induction charger, and wherein each of the plurality of surgical instruments wirelessly communicate with the first adaptor and the second adaptor, and wherein when the plurality of the first halves of the induction charger are coupled to at least one of the second halves of the induction charger, the source of electrical energy charges at least one of the batteries.

9. The electrosurgical system of claim 1, wherein the controller is configured to deliver the determined proper current to the plurality of instruments in use when the distance between the working ends of the plurality of instruments is less than a cutoff distance.

10. The electrosurgical system of claim 9, wherein the cutoff distance is 5 mm.

11. The electrosurgical system of claim 1, wherein the controller is configured to determine a type of the target tissue based on an impedance of the target tissue.

12. The electrosurgical system of claim 11, wherein the controller uses Electrochemical Impedance Spectroscopy (EIS) to determine the type of the target tissue.

13. The electrosurgical system of claim 11, wherein the controller comprises an artificial intelligence and/or machine learning algorithm.

14. The electrosurgical system of claim 1, wherein the sensor is a pressure sensor.

15. The electrosurgical system of claim 1, wherein each of the plurality of surgical instruments comprises a different surgical tool, and wherein the controller is configured to automatically detect each different surgical tool.

16. An electrosurgical system for use in manipulating and cauterizing target tissue, comprising:
a plurality of surgical instruments, wherein each of the plurality of surgical instruments comprises:
at least one sensor; and
a surgical tool at a working end of the surgical instrument, wherein the surgical tool is configured to deliver electrical energy to a target tissue site and to perform an additional function other than delivering the electrical energy to the target tissue site, wherein the additional function comprises manipulating tissue; and
a source of electrical energy configured to be electrically coupled to the plurality of surgical instruments, the source of electrical energy comprising a controller, wherein the controller is configured to determine a desired operating condition based on a size, a shape or a type of the surgical instruments coupled to the source of electrical energy and/or based on information received from the at least one sensor.

17. The electrosurgical system of claim 16, wherein the at least one sensor comprises an accelerometer and/or a gyroscope.

18. The electrosurgical system of claim 17, wherein the at least one sensor is configured to detect when the surgical instrument is in use.

19. The electrosurgical system of claim 16, further comprising an adaptor, wherein the adaptor is configured to couple the plurality of surgical instruments to the source of electrical energy.

20. The electrosurgical system of claim 19, wherein the adaptor comprises the controller.
